(19) United States
(12) Patent Application Publication
(10) Pub. No.: US 2007/0288016 A1
(43) Pub. Date: Dec. 13, 2007

(54) BONE SUPPORT

(75) Inventor: Subhash Chandra Halder, Halifax (GB)

Correspondence Address: MICHAEL BEST & FRIEDRICH LLP, 100 E WISCONSIN AVENUE, Suite 3300, MILWAUKEE, WI, 53202, US

(73) Assignee: Subhash Chandra Halder, Halifax (GB)

(21) Appl. No.: 11/422,207

(22) Filed: Jun. 5, 2006

(51) Int. Cl. A61F 2/30

(52) U.S. Cl. 606/62

(57) ABSTRACT

A bone support is provided comprising: a hollow elongate rod 10 having trailing and leading ends; said hollow rod being insertable along at least a substantial part of the length of the interior of a bone; an elongate fixing device 14 positioned within said hollow rod 10, the elongate fixing device 14 having trailing and leading ends, the leading end of said elongate fixing device 14 having a plurality of projections 17, 18, 19 which can be extended out of the rod to engage with bone surrounding the leading end of the rod, thus acting as a bone rotation prevention means; said elongate fixing device being operable from the trailing end of the rod; the leading end of the rod having side apertures 20; and the bone support including an end plug 13 having inner and outer ends; the inner end of the plug having inwardly tapered surfaces 26 adjacent to said side apertures 20 which surfaces engage with the projections of the elongate fixing device when the fixing device is moved within the rod, towards the leading end of the rod, engagement of the projections 17, 18, 19 with the tapered surfaces 26 guiding the projections out of said side apertures 20, and causing the projections to splay outwardly and penetrate bone surrounding the leading end of the rod.

### FIELD OF INVENTION

The invention relates to apparatus for the support of a fractured or otherwise damaged bone, for example the humerus, tibia or femur.

In recent years, there has been an increasing tendency to treat fractures of bones of limbs by supporting the bone itself internally rather than by supporting the limb as a whole externally with a splint or plaster cast.

For example, known apparatus for the support of a fractured bone typically comprises an elongate, hollow rod, commonly referred to as a “nail”. In use, the nail is inserted into the medullary canal of the bone through an opening made in one end of the bone. In order to prevent rotational movement of parts of the bone at either side of the fracture relative to one another, the nail is locked in position at each end.

The invention relates to an improved form of bone support.

### BRIEF DESCRIPTION OF THE PRIOR ART

An earlier form of bone support invented by me is disclosed in EP 0 738 502.

In this earlier invention, rotational movement is prevented at the leading end by portions of a projection member movable within the elongate hollow rod. This causes the projections to splay out and engage with bone surrounding the support.

U.S. Pat. No. 5,603,715 (Kessler) discloses another embodiment of support in which separate wires are caused to splay out to engage bone. In Kessler the separate wires are guided by curved paths formed in a terminal cap. This cap is relatively complicated to manufacture and separate passages have to be constructed within the cap for each wire. Furthermore, although in the Kessler device it is relatively easy to insert the first wire, it is harder then to insert the second wire, for example because of possible obstruction by the first wire. As there are two different wires to control, this makes the procedure more complex for the surgeon, Another problem with the Kessler device is that the individual wires can twist, one with respect to the other, and there may not be sufficient rigidity in the device to adequately control the tendency of bone fragments to rotate, one with respect to the other.

### PRINCIPAL OBJECT OF THE INVENTION

The principal object of the invention is to provide a bone support which is easier to manufacture than known bone supports and is more reliable in surgical operation, and which makes it much easier to carry out the surgical procedure.

### BRIEF SUMMARY OF THE INVENTION

The invention is suitable for use with fractures of the tibial bone, humeral bone, or femural bone.

According to one aspect, the present invention provides a bone support comprising: a hollow elongate rod, said hollow elongate rod having trailing and leading ends; said hollow rod being insertable along at least a substantial part of the length of the interior of a bone; an elongate fixing device positioned within said hollow rod, the elongate fixing device having trailing and leading ends, the leading end of said elongate fixing device having a plurality of projections which can be extended out of the rod to engage with bone surrounding the leading end of the rod, thus acting as a bone rotation prevention means; said elongate fixing device being operable from the trailing end of the rod; the leading end of the rod having side apertures; and the bone support including an end plug having inner and outer ends; the inner end of the plug having inwardly tapered surfaces adjacent to said side apertures which surfaces engage with the projections of the elongate fixing device when the fixing device is moved within the rod, towards the leading end of the rod, engagement of the projections with the tapered surfaces guiding the projections out of said side apertures, and causing the projections to splay outwardly and penetrate bone surrounding the leading end of the rod.

It is relatively simple to manufacture the plug with the tapered surfaces and fasten this into the leading end of the hollow rod, for example by welding. Alternatively the plug could be a press fit, crimped, glued or screwed.

It is very simple for the surgeon to bring about this movement of the projections, to penetrate bone surrounding the leading end of the rod, and a surgeon only has to control the movement of the elongate fixing device.

A plug is preferably manufactured from a cylindrical piece of metal, one end of which is shaped to provide the inwardly tapered surfaces.

Preferably there are three inwardly tapered surfaces evenly spaced apart around the plug, the elongate fixing device being provided with three projections which, in use, engage respectively with the three inwardly tapered surfaces.

Preferably the plug is elongate and has a through bore extending along the longitudinal axis of the plug, to enable the bone support to be used with a guide wire.

Preferably the plug has at least one transverse bore to receive a bone fixing screw if necessary.

Preferably the trailing end of the rod has at least one transverse through opening to receive a bone fixing screw if necessary.

The through opening may take the form of a slot,

Preferably the elongate fixing device comprises first, second and third wires, welded together.

Preferably the first wire has a greater length than the second and third wires.

The second wire may have a greater length than the third wire.

In an alternative embodiment the first and second wires are equal in length and both have a greater length than the third wire.

Preferably the elongate fixing device terminates in an enlarged head.

The hollow elongate rod may be provided with a screw threaded closure cap to close the trailing end of the rod.

According to a further aspect, the present invention provides a bone support comprising: a hollow elongate rod said hollow elongate rod having trailing and leading ends; said hollow rod being insertable along at least a substantial part of the length of the interior of a bone; an elongate fixing device positioned within said hollow rod, the elongate fixing device having trailing and leading ends; the leading end of said elongate fixing device having a plurality of projections which can extend out of the rod to engage with bone surrounding the leading end of the rod, thus acting as a bone rotation prevention means; said elongate fixing device being operable from the trailing end of the rod; the leading end of the rod having side apertures; the bone support having engagement means adjacent to said side apertures; which engagement means engage with the projections of the elongate fixing device when the fixing device is moved within the rod, towards the leading end of the rod; engagement of the projections with the engagement means causing the projections to splay outwardly with the help of an end plug and penetrate bone surrounding the leading end of the rod; said hollow elongate rod having a transverse aperture in the region of its trailing end; the transverse dimension of the elongate fixing device becoming smaller towards the trailing end of the elongate fixing device; the reducing transverse dimension of the elongate fixing device facilitating the insertion of at least one fixing screw through said transverse aperture of said hollow rod.

Further objects and advantages of the present invention will be apparent from the following description of the preferred embodiment.

### BRIEF DESCRIPTION OF THE DRAWINGS

The invention will be further explained as follows, by reference to a specific embodiment and to the following drawings:

FIG. 1 is a side view, shown in exploded form, of an embodiment of bone support according to the invention;

FIG. 2 is a longitudinal cross-sectional view of the hollow rod of the embodiment of FIG. 1 aligned with a trailing end closure cap;

FIG. 3 is a view perspective view of a plug for the hollow rod;

FIG. 4 is a longitudinal cross-sectional view of the leading end of the rod, and the internal elongate fixing device, with the projections starting to splay;

FIG. 5 is a view similar to FIG. 5 but showing the projections in a more splayed out position; and

FIG. 6 is a side view of an alternative embodiment in use with a fracture of a humerus.

### DESCRIPTION OF PREFERRED EMBODIMENTS

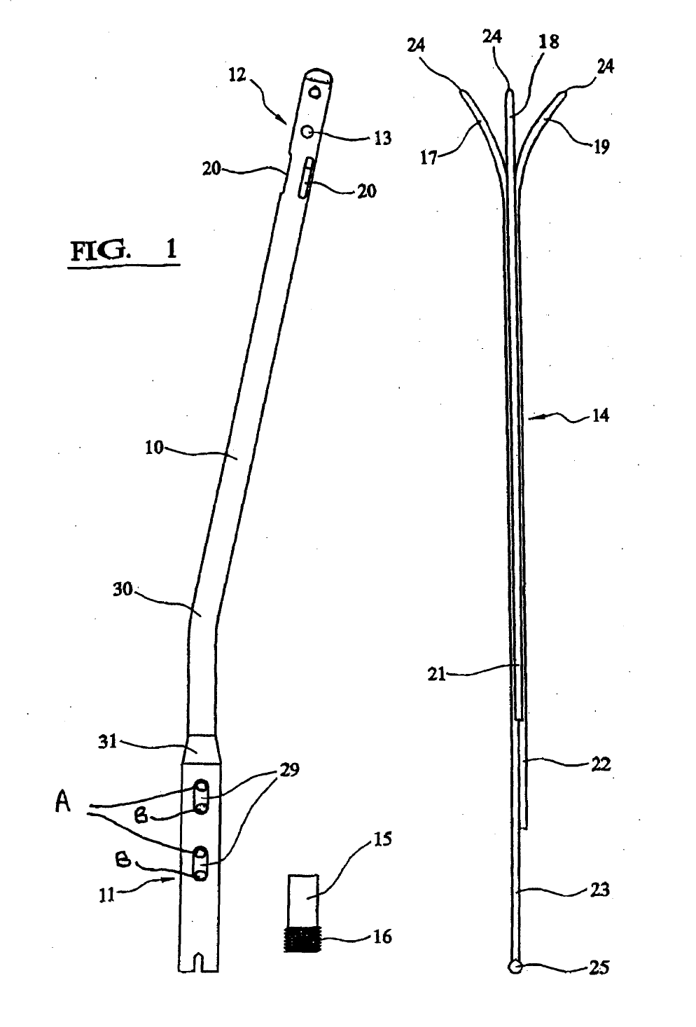

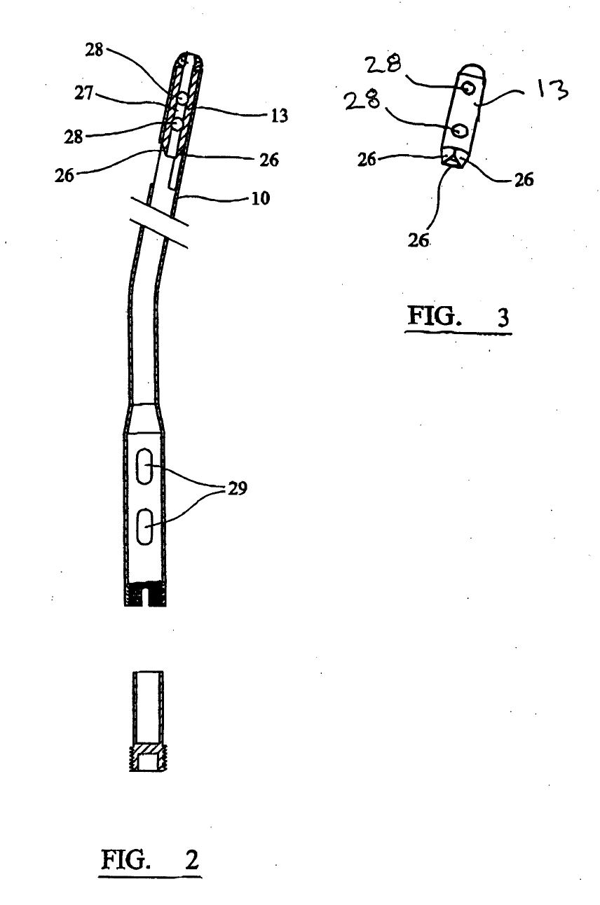

FIG. 1 shows the components of a bone support in the form of a nail for use in supporting a fractured or otherwise damaged tibial bone.

The device shown in FIG. 1 comprises four components, all manufactured from stainless steel. Firstly there is a hollow elongate rod 10 which has a trailing end 11 and a leading end 12.

At the leading end 12 there is a separately constructed plug 13 which will be described in more detail below. The plug 13 is welded into the leading end of the rod 10.

A third component comprises an elongate fixing device 14. In use this device is positioned within the rod 10 but in FIG. 1 the elongate fixing device 14 is shown separately for clarity.

Once the elongate fixing device 14 has been inserted into the rod 10 and positioned as desired during surgery, the trailing end 11 of the hollow rod 10 may be closed by a closure cap 15 which has a screw threaded portion 16 which can engage with internal screw threads within the trailing end of the rod 10. The cap ensures accurate positioning of the fixing device 14, reducing the risk of post-operative migration of the fixing device 14 and the cap also prevents bony in-growth.

The bone support is used in a similar manner to that described in EP 0 738 502 and a detailed explanation of the operating technique is not therefore necessary.

The elongate fixing device 14 has three projections 17, 18 and 19. The objective when using the bone support is to arrange for the projections 17, 18 and 19 to be moved out through three elongate apertures 20 in the hollow rod 10, so that these projections penetrate bone surrounding the leading end of the bone support, to prevent rotation for the same reasons as described in EP 0 738 502.

The elongate fixing device 14 is constructed by welding together three stainless steel wiring 21, 22 and 23, as can clearly be seen in the right hand part of FIG. 1.

The leading end of each wire is identical, with a pointed, but not sharp, end 24.

The wires are pre-sprung, flared and strong.

However, the rods have differing lengths, so that as one moves along the elongate fixing device, from the leading to the trailing end, the transverse dimension of the elongate fixing device steadily reduces, until it consists of the single wire 23 at the trailing end. The purpose of this reducing dimension, which forms one important aspect of the invention will be described in more detail below.

The wire 23 terminates in an enlarged head 25 which facilitates manipulation of the device.

A primary feature of this invention concerns the plug 13. The plug 13 is fitted into the leading end of the rod 10 and it is secured there by welding. The plug 13 can readily be machined from a short cylinder of stainless steel. The inner end of the plug is shaped to provide three surfaces 26 which taper inwardly. The three surfaces are evenly spread apart around the longitudinal axis of the plug. These surfaces coincide with the three elongate apertures 20. The plug also has a central bore 27 and two transverse apertures 28.

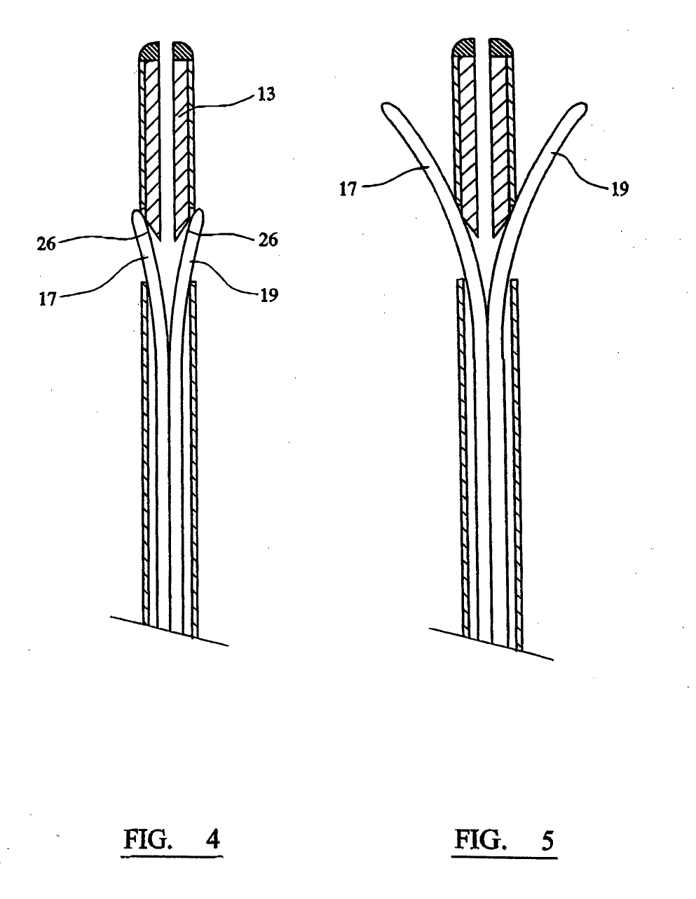

In use, once the rod 10 has been inserted along at least a substantial part of the length of the interior of the bone to be supported, the elongate fixing member 14 is manipulated, to bring the projections 17, 18, 19 into engagement with the tapered faces 26 of the plug 13. This helps to push the ends 24 of the elongate fixing device 14 outwardly through the elongate apertures of the hollow rod. This is best seen in FIG. 4, where the projection 18 has been omitted for clarity.

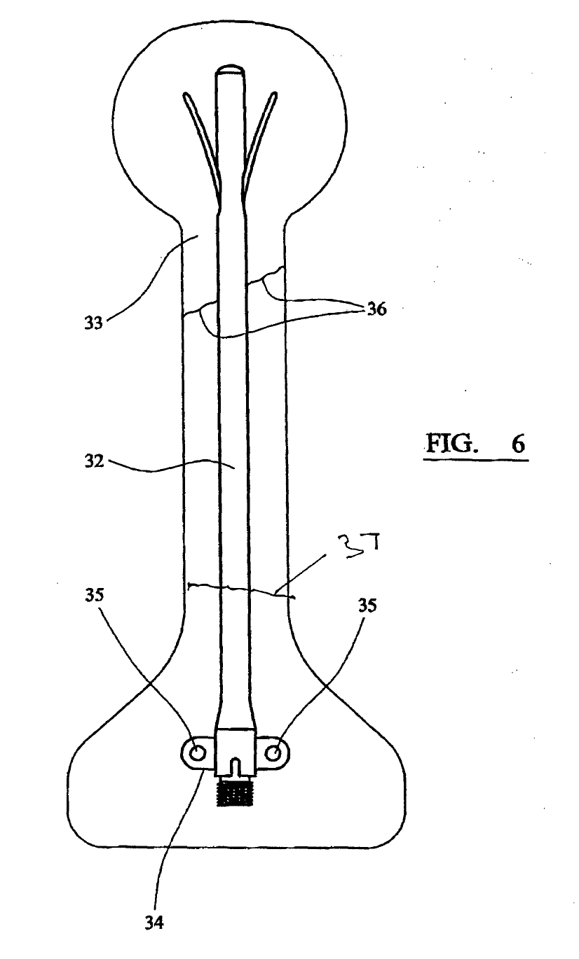

The faces 26 provide a simply constructed but very effective and positive way of guiding the projections in their desired direction. As movement of the elongate fixing device is continued in the leading direction, the projections gradually splay out more, but in a predictable and controlled manner, as shown in FIG. 5. The surgeon has only to control one component, instead of several separate wires as in the Kessler device.

The cross apertures 28 enables the surgeon to insert one or two additional fixing screws if necessary in a complicated case, to further pin together the bone and the support.

Further cross apertures 29, this time elongate in shape, are provided in the hollow rod, and this enables one or possibly two fixing screws to be inserted, to restrain rotation movement of the bone support with respect to the surrounding bone.

By arranging for the elongate fixing device 14 to reduce its transverse dimension towards the trailing end of the bone support, more space is provided for screws to pass through the transverse bore 29 without fouling on the elongate fixing device.

The invention may include other features and variations, without departing from the scope of the following claims. For example, the embodiment shown in the figures may be adapted for use in supporting the femoral or humeral bone, rather than only the tibial bone. The bone support shown in the figures is particularly suitable for use with tibial fractures, being provided with a slight bend 30, which does not preclude passage of the elongate fixing device 14, since the wires are flexible, and the hollow rod 10 is also provided with a tapered portion 31 which facilitates entry of the rod into the bone to be supported.

In some complicated fractures, where one section of bone has moved out of register with another section of bone, in a direction transverse to the longitudinal axis of the bone, it may be necessary for the surgeon first to insert a guide wire to align the bones, and this guide wire can also be used to take measurements to decide which length of bone support should be utilised. It is desirable to leave this guide wire in place within the bone, while the bone support is being inserted. This is made possible by the central bore 27 of the plug 13 and the hollowness of the rod 10. As the bone support is inserted into the bone, it slides along the length of the guide wire and once the bone support is in position, the guide wire can be withdrawn.

Although the bone support has to be inserted through a significant length of the bone to be supported, the fact that the support is simple to manipulate, and has few moving parts, means that there is no need for X-ray control for insertion of the elongate fixing device 14, to restrain any rotation of the distal fragment (with the femur or tibia), and to restrain rotation of the proximal fragment with the humerus Known apparatus needs X-ray control when placing known fixing devices. Since the device according to the invention does not require X-ray control there is a significant reduction in radiation to the hands of the surgeon during the operation. There is also a time saving which also benefits the operating theatre, nurses and anaesthetists.

The fixing device according to the invention also makes it possible to dynamise (as it is known) the fracture. This involves putting the bone under compression, while at the same time restraining rotation of the broken fragment. The compression comes about as a result of reaction forces when the projections 17, 18 and 19 are projected into the bone. Because the projections move at an angle, and not purely parallel to, or transverse to, the longitudinal axis of the device, there is a component of force which brings about the desired compression. This dynamisation helps with early healing of the fracture for its micro movement in the direction of the longitudinal axis of the device.

When it is desired to dynamise the fracture, cross screws are inserted through the elongate cross apertures 29 at the positions B shown in FIG. 1. This allows the bone fragment to move in the direction of the longitudinal axis of the device, to bring about the compression. If dynamisation is not required, and the bone fragment is to be statically pinned, then screws are inserted at the points A in FIG. 1.

The device according to the invention also makes it possible to treat extreme distal fracture in the case of the tibia or femur, and extreme proximal and distal fracture in the case of the humerus. It is normally difficult to fix this kind of fracture because it is normally essential to use cross screws, and there can be insufficient space to insert even one cross screw, with this type of extreme fracture.

FIG. 6 shows an embodiment of the invention 32 for use with a fractured humerus 33 having a fracture 36. The embodiment for use with the humerus 33 is substantially straight, to suit the different shape of that particular bone, and this embodiment is also provided with a cross bracket 34 which can be pinned to the bone by inserting fixing through screws through apertures 35. The projecting ends of the bracket 34 lie against the adjacent bone, virtually eliminating any tendency for that end of the rod to rotate with respect to the surrounding bone. With the help of this cross bracket 35 the most distal fracture, for example as indicated by reference 37 can be treated effectively. Whereas other intramedullar devices have problems in dealing with such fractures.

The invention is not restricted to the details of the foregoing embodiments. For example, the wire 22 shown in FIG. 1 could be made the same length as wire 23. Titanium alloy could be used instead of stainless steel. Reinforced plastics or any orthopaedic metal or polymer could be used. The embodiments of the invention have superior strength, and reliability compared to prior art devices. Fixation of fractures is particularly reliable and the devices are very effective throughout rehabilitation of patients.

1. A bone support comprising: a hollow elongate rod, said hollow elongate rod having trailing and leading ends; said hollow rod being insertable along at least a substantial part of the length of the interior of a bone; an elongate fixing device positioned within said hollow rod, the elongate fixing device having trailing and leading ends, the leading end of said elongate fixing device having a plurality of projections which can be extended out of the rod to engage with bone surrounding the leading end of the rod, thus acting as a bone rotation prevention means; said elongate fixing device being operable from the trailing end of the rod; the leading end of the rod having side apertures; and the bone support including an end plug having inner and outer ends; the inner end of the plug having inwardly tapered surfaces adjacent to said side apertures which surfaces engage with the projections of the elongate fixing device when the fixing device is moved within the rod, towards the leading end of the rod, engagement of the projections with the tapered surfaces guiding the projections out of said side apertures, and causing the projections to splay outwardly and penetrate bone surrounding the leading end of the rod.

2. A bone support as claimed in claim 1, in which the plug is manufactured separately and is then welded into the leading ends of the hollow rod.

3. A bone support as claimed in claim 1, in which the plug is manufactured from a cylindrical piece of metal, one end of which is shaped to provide the inwardly tapered surfaces.

4. A bone support as claimed in claim 3, in which there are three inwardly tapered surfaces evenly spaced apart around the plug, the elongate fixing device being provided with three projections which, in use, engage respectively with the three inwardly tapered surfaces.

5. A bone support as claimed in claim 1, in which the plug is elongate and has a through bore extending along the longitudinal axis of the plug, to enable the bone support to be used with a guide wire.

6. A bone support as claimed in claim 1, in which the plug has at least one transverse bore to receive a bone fixing screw if necessary.

7. A bone support as claimed in claim 1, in which the trailing end of the rod has at least one transverse through opening to receive a bone fixing screw if necessary.

8. A bone support as claimed in claim 7, in which the through opening may take the form of a slot.

9. A bone support as claimed in claim 1, in which the elongate fixing device comprises first, second and third wires, welded together.

10. A bone support as claimed in claim 9, in which the first wire has a greater length than the second and third wires.

11. A bone support as claimed in claim 10, in which the second wire has a greater length than the third wire.

12. A bone support as claimed in claim 9, in which the first and second wires are equal in length and both have a greater length than the third wire.

13. A bone support as claimed in claim 9, in which the elongate fixing device terminates in an enlarged head.

14. A bone support comprising: a hollow elongate rod said hollow elongate rod having trailing and leading ends; said hollow rod being insertable along at least a substantial part of the length of the interior of a bone; an elongate fixing device positioned within said hollow rod, the elongate fixing device having trailing and leading ends; the leading end of said elongate fixing device having a plurality of projections which can extend out of the rod to engage with bone surrounding the leading end of the rod, thus acting as a bone rotation prevention means; said elongate fixing device being operable from the trailing end of the rod; the leading end of the rod having side apertures; the bone support having engagement means adjacent to said side apertures; which engagement means engage with the projections of the elongate fixing device when the fixing device is moved within the rod, towards the leading end of the rod; engagement of the projections with the engagement means causing the projections to splay outwardly with the help of an end plug and penetrate bone surrounding the leading end of the rod; said hollow elongate rod having a transverse aperture in the region of its trailing end; the transverse dimension of the elongate fixing device becoming smaller towards the trailing end of the elongate fixing device; the reducing transverse dimension of the elongate fixing device facilitating the insertion of at least one fixing screw through said transverse aperture of said hollow rod.

15. A bone support as claimed in claim 14, in which the elongate fixing device comprises a plurality of wires secured together, the reduction in transverse dimension being achieved by using wires which differ in length.

16. A bone support as claimed in claim 1, in which the trailing end of the rod is provided with a cross bracket which can be pinned to the bone being treated by inserting fixing screws through apertures in the bracket.

17. A bone support as claimed in claim 14, in which the trailing end of the rod is provided with a cross bracket, which can be pinned to the bone being treated by inserting fixing screws through apertures in the bracket.
